(19) United States
(12) Patent Application Publication
(10) Pub. No.: US 2024/0122874 A1
(43) Pub. Date: Apr. 18, 2024

(54) ORAL ANTI-RADIATION MICROALGAE-NANOPARTICLE COMPOUND PREPARATION, AND PREPARATION METHOD AND USE THEREOF

(71) Applicant: ZHEJIANG UNIVERSITY, Hangzhou (CN)

(72) Inventors: Min Zhou, Hangzhou (CN); Dongxiao Zhang, Hangzhou (CN); Jian He, Hangzhou (CN)

(21) Appl. No.: 18/242,697

(22) Filed: Sep. 6, 2023

(30) Foreign Application Priority Data
Oct. 17, 2022 (CN) 202211269196.5

(51) Int. Cl. A61K 31/122; A61K 9/00; A61K 47/61; A61K 47/69; A61P 39/06

(52) U.S. Cl. CPC A61K 31/122; A61K 9/0053; A61K 47/61; A61K 47/6901; A61K 47/6937; A61P 39/06

(57) ABSTRACT

Provided are an oral anti-radiation microalgae-nanoparticle compound preparation, and a preparation method and use thereof. The oral anti-radiation microalgae-nanoparticle compound preparation, including a microalgae, an anti-radiation drug, and nanoparticles; wherein the anti-radiation drug is loaded on the nanoparticles to form drug-loaded nanoparticles, and the drug-loaded nanoparticles are loaded on a surface of the microalgae through a surface modifier to form the oral anti-radiation microalgae-nanoparticle compound preparation for direct oral administration.

### CROSS REFERENCE TO RELATED APPLICATION

This patent application claims the benefit and priority of Chinese Patent Application No. 2022112691965 filed with the China National Intellectual Property Administration on Oct. 17, 2022, the disclosure of which is incorporated by reference herein in its entirety as part of the present application.

### TECHNICAL FIELD

The present disclosure belongs to the technical field of biomedicine, and relates to an oral anti-radiation microalgae-nanoparticle compound preparation, and a preparation method and use thereof.

### BACKGROUND

Human exposure to ionizing radiation is increasing as radioactive substances and ionizing radiation are widely used in various aspects of modern society, including nuclear energy, diagnostic radiology, radiation therapy, agriculture, and industry. Despite its various beneficial applications, ionizing radiation can also cause damages to people's health. In situations such as tumor radiotherapy or nuclear accidents, radiation-sensitive organs may be exposed, causing a series of structural damages and dysfunction. Intestinal radiation injury is a common and serious adverse reaction in tumor radiation therapy. Due to the large volume and area of small intestine and its high sensitivity to radiation, radiation damages to the small intestine are generally unavoidable and difficult to treat. In accidents such as nuclear accidents, the whole body of a person may be radiated, resulting in systemic damages, multiple organ lesions, and even death. At present, effective drugs with radiation protection effects are still lacking, especially convenient oral preparations. Therefore, it is of great significance to develop drugs, drug complexes, or preparations for preventing radiation-induced intestinal and systemic damages.

Since the generation of reactive oxygen species (ROS) is an important mechanism of radiation-induced cellular damages, many antioxidants have been developed as radioprotectants. For example, astaxanthin (ASX), as one of the strongest natural-source antioxidants, has a high ability to scavenge oxygen free radicals, and can reduce the generation of ROS and cell damages caused by radiation. In addition, the ASX has anti-inflammatory, immune regulation, and intestinal flora regulation functions. However, ASX has the disadvantages of poor solubility in water and weak stability in presence of acid, oxygen, heat, and light, and shows low oral bioavailability, making it difficult to function by oral administration. The strategy of micro- or nano-encapsulation for the ASX can improve the water solubility and stability, and is beneficial to increase the oral absorption rate and bioavailability.

In summary, the present disclosure provides a strategy of a combination of natural microalgae and nanoparticles to overcome the intractable radiation damages.

### SUMMARY

An object of the present disclosure is to provide an oral anti-radiation microalgae-nanoparticle compound preparation, and a preparation method and use thereof. The anti-radiation compound preparation is a microalgae-nanoparticle composite system loaded with a drug showing anti-radiation functions, in which microalgae is combined with nanoparticles loaded with an anti-radiation drug. The compound preparation makes it possible to improve water-solubility and oral absorbability of the anti-radiation drug, and realize long-term retention in the intestinal tract, multi-stage slow release of the drug, and gradual degradation, which effectively enhances distribution and bioavailability of the drug in the intestinal tract, and has a beneficial regulatory effect on intestinal flora and metabolites thereof, thereby providing effective radiation protection for the intestinal tract and the whole body.

To achieve the above object, the present disclosure adopts the following technical solutions: an oral anti-radiation microalgae-nanoparticle compound preparation, and a preparation method and use thereof. The technical solutions are specifically described as follows:

The present disclosure provides an oral anti-radiation microalgae-nanoparticle compound preparation, including the following components in percentages by mass: 30 wt % to 90 wt % of a microalgae, 1 wt % to 30 wt % of anti-radiation drug-loaded nanoparticles, and 1 wt % to 30 wt % of a surface modifier for nanoparticles. The anti-radiation drug includes one or more components that prevent and/or treat radiation-induced cell, tissue, or organ damages, such as astaxanthin, resveratrol, vitamin E, lycopene, zeaxanthin, curcumin, epigallocatechin gallate, and amifostine. The microalgae-nanoparticle compound preparation is universally applicable to loading of the above drugs, and can achieve effective loading especially for poorly water-soluble or water-insoluble drugs such as astaxanthin. In contrast, when only microalgae is used as a carrier, a poor loading effect of the poorly water-soluble or water-insoluble drugs is shown. The microalgae includes but not limited to natural microalgae such as Spirulina, Haematococcus pluvialis, Chlorella, Euglena, and Chlamydomonas reinhardtii.

The present disclosure also provides a method for preparing the oral anti-radiation microalgae-nanoparticle compound preparation as described in the above technical solutions, including: mixing a nano-carrier raw material (such as nanoparticles) with the anti-radiation drug in a solution, such that the carrier raw material encapsulates the anti-radiation drug through interaction to obtain the anti-radiation drug-loaded nanoparticles; adding the surface modifier into a first resulting solution containing the drug-loaded nanoparticles, and performing reaction for a period of time to obtain surface-modified drug-loaded nanoparticles, such that the nanoparticles is able to bind with a microalgae; and adding the microalgae into a second resulting solution containing the surface-modified drug-loaded nanoparticles, and mixing to obtain the oral anti-radiation microalgae-nanoparticle compound preparation, in which the microalgae and the drug-loaded nanoparticles are combined.

In some embodiments, the nanoparticles include at least one selected from the group consisting of poly(lactic-co-glycolic acid) (PLGA) nanoparticles, liposome nanoparticles, porous silica nanoparticles, porous carbon nanoparticles, dendrimer nanoparticles, and metal nanoparticles.

In some embodiments, the surface modifier for nanoparticles is one or more biomedical polymer materials selected from the group consisting of chitosan, polyethyleneimine, guar gum, dopamine, sodium hyaluronate, polyvinyl alcohol, sodium alginate, calcium alginate, gelatin, and a cellulose derivative. Materials that can provide positive charges, such as the chitosan, are preferred. Under the condition that the chitosan is used as the surface modifier, surfaces of the nanoparticles are positively charged, and could combine with negatively charged microalgae through charge adsorption to construct a microalgae-nanoparticle compound preparation. Meanwhile, the positively-charged surface can make the nanoparticles more easily absorbed by cells.

In some embodiments, the anti-radiation drug-loaded nanoparticles have a nano-scale particle size.

In some embodiments, the oral anti-radiation microalgae-nanoparticle compound preparation further includes a pharmaceutically acceptable excipient.

In some embodiments, the solution includes but is not limited to a single solution or a mixed solution of a water-soluble solution such as water, a phosphate buffer, a citrate buffer, an acetate buffer, and a tris(hydroxymethyl) aminomethane hydrochloride (Tris-HCl) buffer; a polar and non-polar solution under synthetic conditions such as dichloromethane, acetone, ethanol, and methanol.

The present disclosure further provides an oral anti-radiation medicine, including the oral anti-radiation microalgae-nanoparticle compound preparation as described in the above technical solutions, wherein the anti-radiation refers to prevention, treatment, or alleviation of a damage or a disease in intestinal tract and other organs of a whole body caused by ionizing radiation or a radioactive substance.

Compared with the prior art, some embodiments of the present disclosure have the following advantages and effects:

An advantage of some embodiments of the present disclosure is to provide an oral anti-radiation microalgae-nanoparticle compound preparation and a preparation method thereof. The anti-radiation preparation is a microalgae-nanoparticle compound system loaded with a drug showing anti-radiation functions, and makes it possible to improve water solubility and absorbability of the drug. This preparation protects a drug activity in the stomach, and has characteristics of multi-stage slow drug release, long intestinal retention time, and wide distribution, which effectively improves the enrichment, absorption, and utilization of the drug in the intestinal tract, and helps the drug exert a radiation protection effect on the intestinal tract and the whole body. In addition to functioning as a carrier, the microalgae also has the effects of supplementing nutrition, enhancing immunity, and anti-inflammation. Moreover, the microalgae can exert a beneficial regulatory effect on the intestinal flora and metabolites thereof, and have an auxiliary effect on the prevention or treatment of radiation damages.

The oral anti-radiation microalgae-nanoparticle compound preparation according to the present disclosure overcomes the following technical difficulties: 1) Many anti-radiation drugs have poor solubility in water and weak stability in the presence of acid, oxygen, heat, and light. On one hand, such drugs are difficult to be absorbed by intestinal cells; and on the other hand, these drugs are easily damaged by gastric acid and digestive enzymes in the stomach and lose their activities, making it difficult to take their effects when administrated orally. 2) The anti-radiation drug enters the intestinal tract after being administrated orally, and is easily diluted or washed away by intestinal fluid. As a result, the drug has a short intestinal residence time and poor intestinal distribution, resulting in low bioavailability and poor oral efficacy. 3) Commonly used orally-available drug carriers/delivery materials generally only play the role of drug delivery and lack of auxiliary therapeutic effects on the disease itself. Moreover, these carriers/delivery materials themselves may have defects such as being difficult to degrade, unable to be absorbed and utilized, or difficult to prepare/extract. Accordingly, in the present disclosure, the nanoparticles are used to encapsulate the anti-radiation drug, such that the drug has desirable water solubility, absorbability, and biocompatibility, and is protected from destruction by gastric acid or digestive enzymes after oral administration. The surface modification of the drug-loaded nanoparticles makes it possible to combine the nanoparticles with the microalgae through interaction to form the microalgae-nanoparticle compound preparation. In the oral anti-radiation microalgae-nanoparticle compound preparation, the microalgae with a micron-scale size prolongs the residence time of the drug-loaded nanoparticles in the intestinal tract. The combined drug-loaded nanoparticles can gradually dissociate in the intestinal tract, and slowly release the anti-radiation drug loaded thereon, forming a multi-stage release behavior of the drug. In this way, the enrichment, absorption, and utilization of the drug in the intestinal tract are effectively enhanced, thereby facilitating exerting a radiation protection effect on the intestinal tract and the whole body. The microalgae used is a carrier that is easily available and does not require complicated preparation processes, has a micron-level size and desirable dispersibility, such that it shows a long residence time and a wide distribution area in the intestinal tract. Moreover, the microalgae contains a variety of nutritional ingredients, not only functions as a carrier, but also has the effects of enhancing immunity, anti-inflammation, and anti-cancer. Furthermore, the microalgae is easy to degrade in the intestinal tract, and can also have a beneficial regulatory effect on the intestinal flora and metabolites thereof.

### BRIEF DESCRIPTION OF THE DRAWINGS

FIG. 1 shows a transmission electron microscopy (TEM) image of the astaxanthin-loaded nanoparticles having surfaces modified with chitosan in Example 1.

FIG. 2 shows hydration particle size distribution of the astaxanthin-loaded nanoparticles having surfaces modified with chitosan in Example 1.

FIG. 3A shows scanning electron microscopy (SEM) images of the anti-radiation microalgae (Spirulina)-nanoparticle compound preparation in Example 1, illustrating that a surface of the Spirulina is evenly covered by the astaxanthin-loaded nanoparticles having surfaces modified with chitosan.

FIG. 3B shows a partial enlarged view of the SEM image in FIG. 3A.

FIG. 4 shows an SEM image of the anti-radiation microalgae (Spirulina)-nanoparticle compound preparation in Example 2, showing a smooth surface of the Spirulina, which is not covered by the nanoparticles.

FIG. 5 shows an SEM image of the anti-radiation microalgae (Spirulina)-nanoparticle compound preparation in Example 3, showing that a surface of the Spirulina is covered by a small number of the nanoparticles.

FIG. 6 shows concentrations of astaxanthin in a solution before and after drug loading on Spirulina in Example 4.

FIG. 7 shows potentials of the Spirulina, the astaxanthin-loaded nanoparticles having surfaces modified with chitosan, and the anti-radiation microalgae-nanoparticle compound preparation in Example 1.

FIG. 8 shows astaxanthin contents in the intestinal tissue of the mouse at different time points after gavage of three different materials, measured by gas chromatography-mass spectrometry.

FIG. 9 shows a pathological image (Ki67 immunohistochemical staining) of a cross-section of the intestinal tract on a third day after the mouse is subjected to 10 Gy of X-ray abdominal radiation (except for the normal group) after gavage with different materials, in which black dotted lines indicate intact intestinal crypts (proliferating stem cells), scale bar being 100 μm.

FIG. 10 shows the number of blood leukocytes one week after the mouse is subjected to 10 Gy of X-ray abdominal radiation (except for the normal group) after gavage with different materials, in which groups 1 to 6 represent normal, radiation exposure, radiation exposure+Spirulina, radiation exposure+astaxanthin, radiation exposure+astaxanthin-loaded nanoparticles, and radiation exposure+Spirulina-astaxanthin-nanoparticle compound preparation, respectively; and results are expressed as mean+standard deviation; * indicates statistical difference, *p<0.05, **p<0.01, and ***p<0.001.

### DETAILED DESCRIPTION OF THE EMBODIMENTS

The following further describes the present disclosure in detail with reference to the accompanying drawings and examples.

The present disclosure provides an oral anti-radiation microalgae-nanoparticle compound preparation, which is prepared by a method including
(1) mixing the anti-radiation drug with the nanoparticles in a solution, such that the nanoparticles encapsulate the anti-radiation drug to obtain drug-loaded nanoparticles through an interaction between nanoparticles and the drug after an organic agent is volatilized;
(2) adding the surface modifier into a first resulting solution containing the drug-loaded nanoparticles obtained in step (1), and performing reaction for a period of time, and removing excess unreacted materials by washing, to obtain surface-modified drug-loaded nanoparticles; and
(3) adding the microalgae into a second resulting solution containing the surface-modified drug-loaded nanoparticles obtained in step (2), and performing reaction for a period of time, and removing excess unreacted materials by washing, to obtain the oral anti-radiation microalgae-nanoparticle compound preparation, in which the microalgae and the drug-loaded nanoparticles are combined.

In some embodiments of the present disclosure, the oral anti-radiation microalgae-nanoparticle compound preparation includes the following components in percentages by mass: 30 wt % to 90 wt % of a microalgae, 1 wt % to 30 wt % of anti-radiation drug-loaded nanoparticles, and 1 wt % to 30 wt % of a surface modifier for nanoparticles. In some embodiments, in step (1), a mass ratio of the nanoparticles to the anti-radiation drug ranges from 0.01:1 to 100:1. In some embodiments, in step (1), the solution in which the nanoparticles are mixed with the anti-radiation drug includes but is not limited to a single solution or a mixed solution of a water-soluble solution such as water, a phosphate buffer, a citrate buffer, an acetate buffer, and a Tris-HCl buffer; a polar and non-polar solution under synthetic conditions such as dichloromethane, acetone, ethanol, and methanol. In some embodiments, in step (1), a process for loading the nanoparticle with the drug includes, but is not limited to, common physical and chemical methods such as ultrasonic vibration, stirring, electrostatic adsorption, covalent bond modification, and microemulsion. In some embodiments, in step (1), loading the nanoparticles with the drug is performed at a temperature, including but not limited to 0° C. to 50° C. In some embodiments, in step (1), loading the nanoparticles with the drug is performed for a period of time, including but not limited to 1 s to 72 h.

In some embodiments, in step (2), the reaction (i.e., modifying a surface of the nanoparticles) is conducted at a temperature, including but not limited to 0° C. to 50° C. In some embodiments, in step (2), the reaction (i.e., modifying a surface of the nanoparticles) is conducted for a period of time, including but not limited to 1 s to 72 h.

In some embodiments, in step (3), an order of adding the microalgae includes, but is not limited to, adding before the surface modifier is added, adding simultaneously with the surface modifier, and adding one part and then adding another part simultaneously with the surface modifier. In some embodiments, in step (3), the reaction is conducted at a temperature, including but not limited to 0° C. to 50° C. In some embodiments, in step (3), the reaction is conducted for a period of time, including but not limited to 1 s to 72 h.

In the present disclosure, the anti-radiation drug is firstly loaded on the nano carrier (i.e., nanoparticles) to obtain drug-loaded nanoparticles, and then surface modification is conducted to endow the nanoparticles with desirable water solubility and biocompatibility, thus protecting the drug from the destruction of gastric acid or digestive enzymes after oral administration. Moreover, the surface modification makes it possible to combine the nanoparticles with the microalgae to form the anti-radiation microalgae-nanoparticle compound preparation.

In the present disclosure, the anti-radiation microalgae-nanoparticle compound preparation is a convenient oral micron-/nano-scale composite drug delivery system, and its micron-level size could prolong the residence time of the preparation in the intestinal tract, such that slowly release the anti-radiation drug loaded thereon. In this way, the enrichment, absorption, and utilization of the drug in the intestinal tract are effectively enhanced, thereby facilitating exerting a radiation protection effect on the intestinal tract and the whole body. Compared with other compound preparations in which microcarriers are combined with the nanoparticles, the microalgae, used as a microcarrier, is a carrier that is easily available and does not require complicated preparation processes, has a micron-level size and desirable dispersibility, such that the compound preparation shows a long residence time and a wide distribution area in the intestinal tract. Moreover, the microalgae contains a variety of nutritional ingredients, not only functions as a carrier, but also has the effects of enhancing immunity, anti-inflammation, and anti-cancer. Furthermore, the microalgae is easy to degrade in the intestinal tract, and can also have a beneficial regulatory effect on the intestinal flora and metabolites thereof.

In some embodiments, in the oral anti-radiation microalgae-nanoparticle compound preparation of the present disclosure, the nanoparticles include PLGA, liposome, porous silica, porous carbon, dendrimer, and metal nanoparticles, and preferably includes polylactic acid (PLA) and the liposome, which show excellent enzyme encapsulating ability and biological safety. In some embodiments, the nanoparticles have a particle size of 5 nm to 1,000 nm. In some embodiments, the anti-radiation drug includes one or more components that prevent and/or treat radiation-induced cell/tissue/organ damages, such as astaxanthin, resveratrol, vitamin E, lycopene, zeaxanthin, curcumin, epigallocatechin gallate, and amifostine. Especially for astaxanthin and other poorly water-soluble or water-insoluble drugs, the method of the present disclosure can achieve a better loading effect than using microalgae alone. In some embodiments, the surface modifier is one or more of biomedical polymer materials, such as chitosan, polyethyleneimine, guar gum, dopamine, sodium hyaluronate, polyvinyl alcohol, sodium alginate, calcium alginate, gelatin, and a cellulose derivative. Materials that can provide positive charges, such as the chitosan, are preferred. The chitosan as a surface modifier could make a surface of the nanoparticles positively charged, and combine with negatively charged microalgae through charge adsorption to construct a microalgae-nanoparticle compound preparation. Meanwhile, the positively-charged surface could make the nanoparticles more easily absorbed by cells, and chitosan could open the tight junction protein between intestinal epithelial cells, thereby improving the intestinal permeability and absorption rate of drugs. In some embodiments, the microalgae includes but not limited to natural microalgae such as Spirulina, Haematococcus pluvialis, Chlorella, Euglena, and Chlamydomonas reinhardtii. Preferably, the Spirulina has a large surface area and a long retention time in the intestinal tract, and can provide desirable loading rate and drug release rate. This kind of microalgae could be fully degraded in the intestinal tract, and is rich in polysaccharides and other ingredients to exert beneficial effects on the intestinal flora, thus playing an auxiliary role in the prevention and treatment of intestinal and systemic damages caused by radiation.

In addition, in some embodiments, the oral anti-radiation microalgae-nanoparticle compound preparation of the present disclosure may also contain a pharmaceutically acceptable excipient. The excipient could be used to prepare the nano-scale antibacterial agent into any dosage form suitable for clinical use, including but not limited to powder, suspension, granule, capsule, and tablet, which could be used in the prevention, treatment, or alleviation of damages to the intestinal tract and other organs, including but not limited to those caused by ionizing radiation. Moreover, experiments have confirmed that the oral anti-radiation microalgae-nanoparticle compound preparation has a highly excellent therapeutic effect and can replace existing conventional drugs.

### Example 1

A method for preparing an oral anti-radiation microalgae-nanoparticle compound preparation was performed according to the following procedures.
(1) Astaxanthin and PLGA were dissolved in dichloromethane (a mass ratio of the astaxanthin to the PLGA being 1:10). 2% polyvinyl alcohol aqueous solution was then added thereto, and a resulting mixture was stirred by ultrasonically oscillating with a probe at 4° C. for 5 min, forming an emulsion. The emulsion was stirred at room temperature for 12 h to volatilize the dichloromethane, initially forming astaxanthin-loaded nanoparticles.
(2) A chitosan solution was added to a solution of nanoparticles prepared in step (1), and stirred at room temperature for 12 h. Excess unreacted materials were removed by washing, obtaining astaxanthin-loaded nanoparticles having modified surfaces.
(3) Spirulina was added into the aqueous solution containing the surface-modified drug-loaded nanoparticles obtained in step (2), wherein a mass ratio of the Spirulina to the drug-loaded nanoparticles being 25:1. A resulting mixture was stirred at room temperature for 30 min, and centrifugated. A resulting precipitate was collected, and excess unreacted materials were removed by washing, obtaining the oral anti-radiation microalgae-nanoparticle compound preparation, in which the microalgae and the drug-loaded nanoparticles were combined.

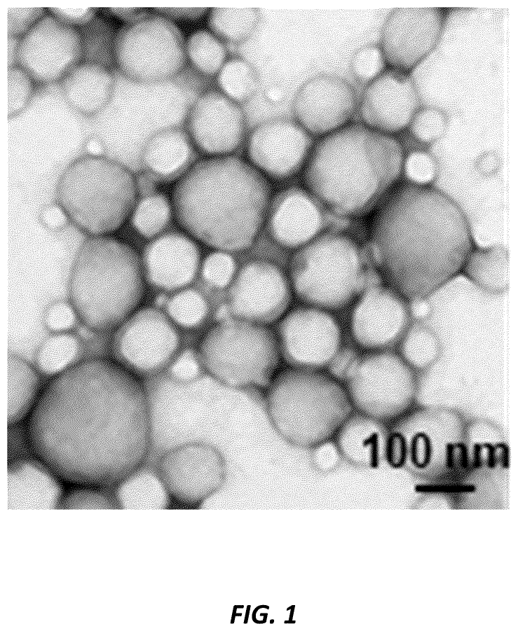

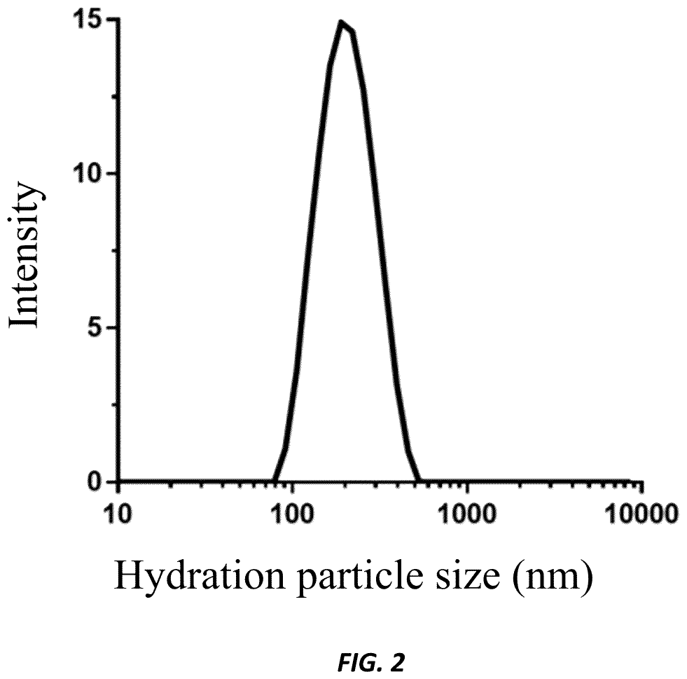

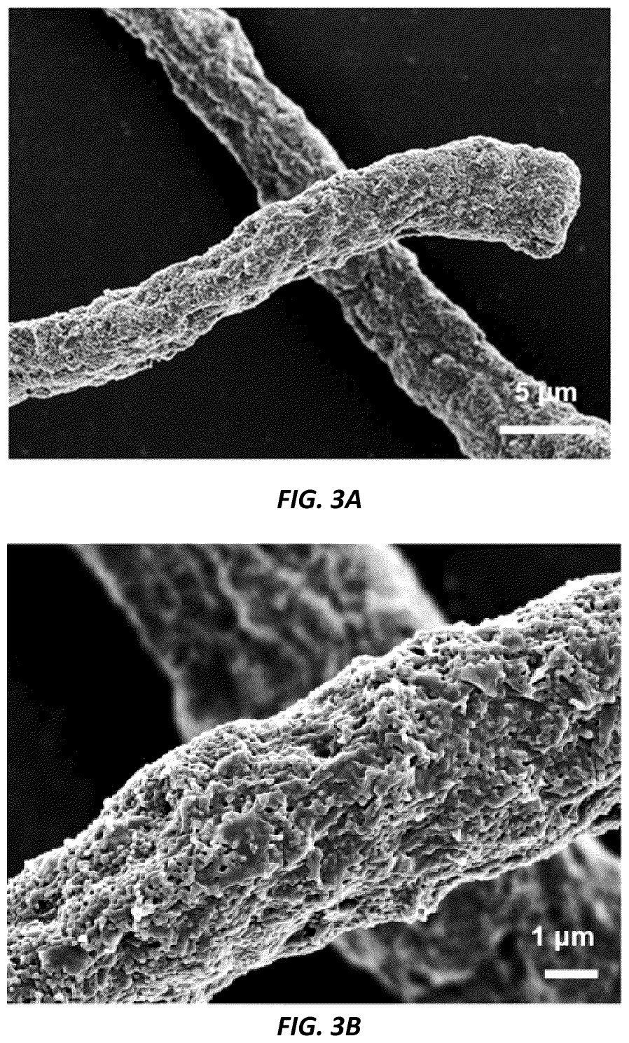

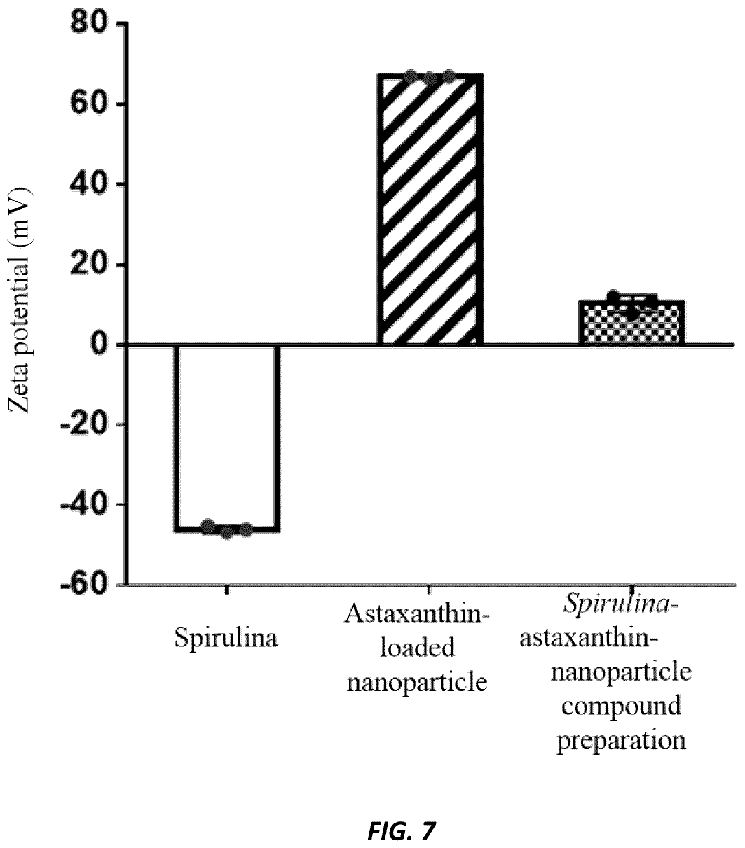

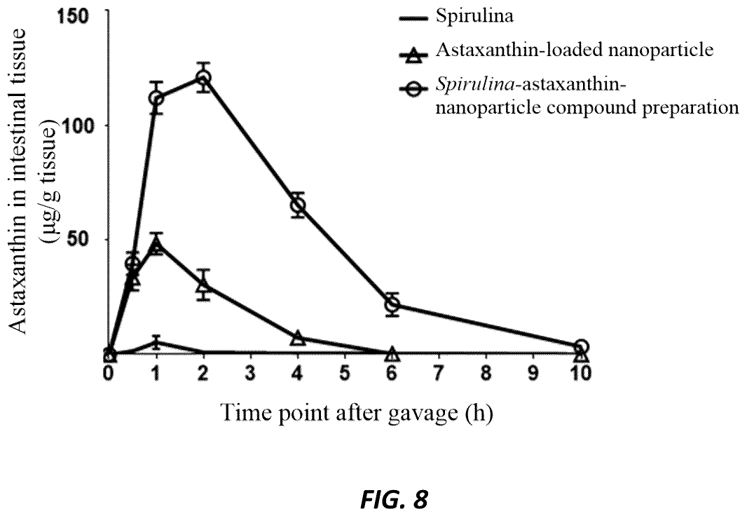

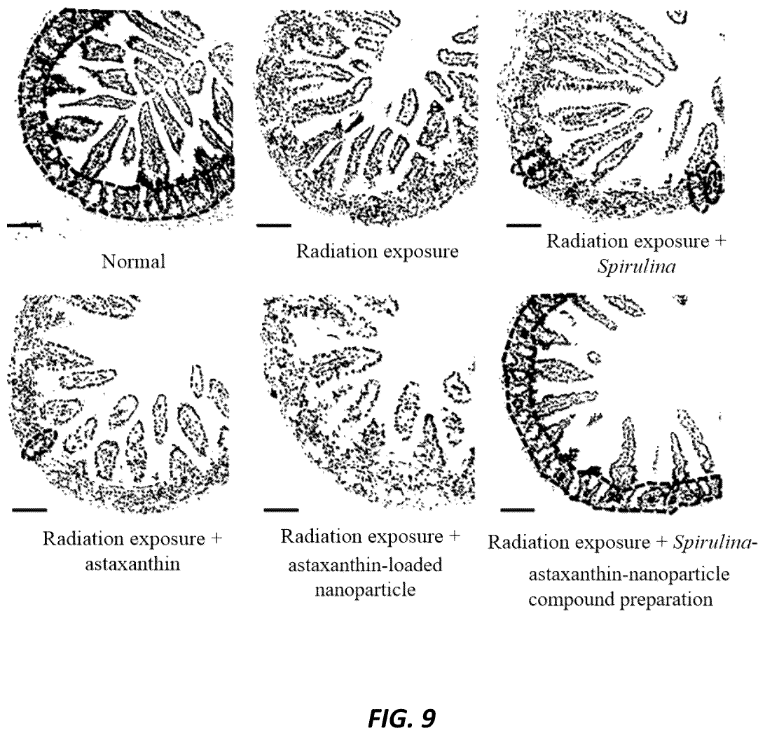

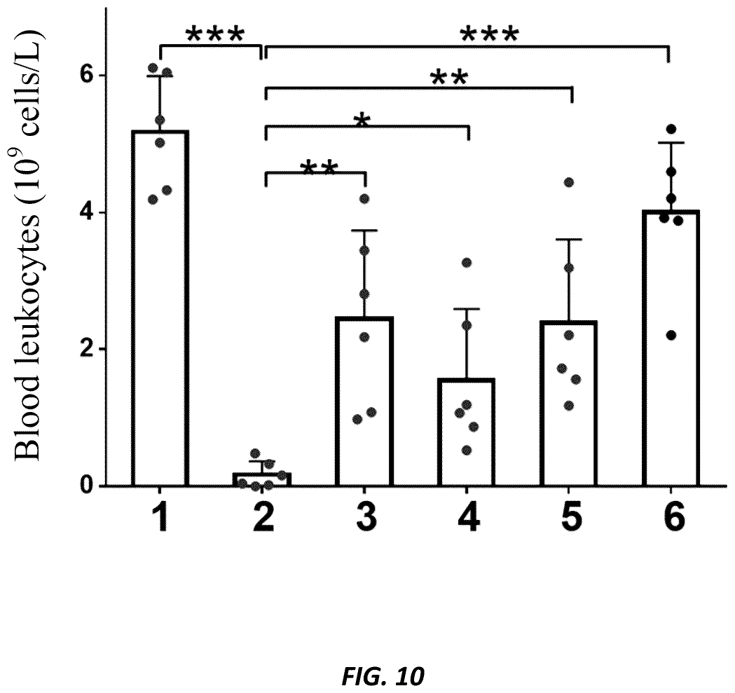

FIG. 1 shows a TEM image of the astaxanthin-loaded nanoparticles having surfaces modified with chitosan in Example 1. FIG. 2 shows hydration particle size distribution, illustrating that the astaxanthin-loaded nanoparticles having surfaces modified with chitosan is a kind of spherical particles with a diameter of about 200 nm. FIGS. 3A and 3B show SEM images of the anti-radiation microalgae (Spirulina)-nanoparticle compound preparation in Example 1 (in which FIG. 3B shows a partial enlarged view of the SEM image in FIG. 3A), illustrating that a surface of the Spirulina is evenly covered by the astaxanthin-loaded nanoparticles having surfaces modified with chitosan. FIG. 7 shows potentials of the Spirulina, the astaxanthin-loaded nanoparticles having surfaces modified with chitosan, and the anti-radiation microalgae-nanoparticle compound preparation in Example 1. As shown in FIG. 7, the surface of the Spirulina is negatively charged, and the astaxanthin-loaded nanoparticles having surfaces modified with chitosan are positively charged; after being combined through charge adsorption, the resulting microalgae-nanoparticle compound preparation has a surface charge between the above two. FIG. 8 shows astaxanthin contents in a mouse intestinal tissue detected by chromatography-mass spectrometry at different time points after administration of three different materials to the mouse by gavage. The results in FIG. 8 show that, compared with astaxanthin and astaxanthin-loaded nanoparticles, the Spirulina-nanoparticle compound preparation could significantly increase the astaxanthin content in intestinal tissue and prolong the drug distribution time, indicating that the compound preparation effectively improves the bioavailability of astaxanthin, with a better effect than that of the two schemes: direct oral administration of astaxanthin and oral administration of astaxanthin-loaded nanoparticles. FIG. 9 shows a pathological image (Ki67 immunohistochemical staining) of a cross-section of the intestinal tract on a third day after the mouse was subjected to 10 Gy of X-ray abdominal radiation (except for the normal group) after gavage with different materials, in which black dotted lines indicate intact intestinal crypts (proliferating stem cells), scale bar being 100 μm. The results in FIG. 9 show that the intact intestinal crypts are largely damaged after being subjected to radiation, while there are still many intestinal crypts in mouse that is subjected to the oral administration of Spirulina-nanoparticle compound preparation, indicating that the oral Spirulina-nanoparticle compound preparation could effectively protect the intestinal tract of the mouse from radiation damages, while other treatment groups have a significantly poor protective effect. FIG. 10 shows the number of blood leukocytes one week after the mouse is subjected to 10 Gy of X-ray abdominal radiation (except for the normal group) after gavage with different materials, in which groups 1 to 6 represent normal, radiation exposure, radiation exposure+spirulina, radiation exposure+astaxanthin, radiation exposure+astaxanthin-loaded nanoparticle, and radiation exposure+Spirulina-astaxanthin-nanoparticle compound preparation, respectively; and results are expressed as mean+standard deviation; * indicated statistical difference, *p<0.05, **p<0.01, and ***p<0.001. FIG. 10 shows that, the number of blood leukocytes in the radiated mice is drastically reduced and the immune function is damaged; oral administration of the Spirulina-nanoparticle compound preparation effectively maintains the number of blood leukocytes in mice and effectively prevent the damage to the blood system of mice caused by radiation. In contrast, other treatment groups have less protective effects.

### Example 2

A method for preparing an oral anti-radiation microalgae-nanoparticle compound preparation was performed according to the following procedures
(1) Astaxanthin and PLGA were dissolved in dichloromethane (a mass ratio of the astaxanthin to the PLGA being 1:10). 2% polyvinyl alcohol aqueous solution was then added thereto, and a resulting mixture was stirred by ultrasonically oscillating with a probe at 4° C. for 5 min, forming an emulsion. The emulsion was stirred at room temperature for 12 h to volatilize the dichloromethane, initially forming astaxanthin-loaded nanoparticles.
(2) Spirulina was added into the aqueous solution containing the drug-loaded nanoparticles having no modified surface obtained in step (1), wherein a mass ratio of the Spirulina to the drug-loaded nanoparticles being 10:1. A resulting mixture was stirred at room temperature for 12 h, and centrifugated. A resulting precipitate was collected, and excess unreacted materials were removed by washing, obtaining the oral anti-radiation microalgae-nanoparticle compound preparation, in which the microalgae and the drug-loaded nanoparticles were combined.

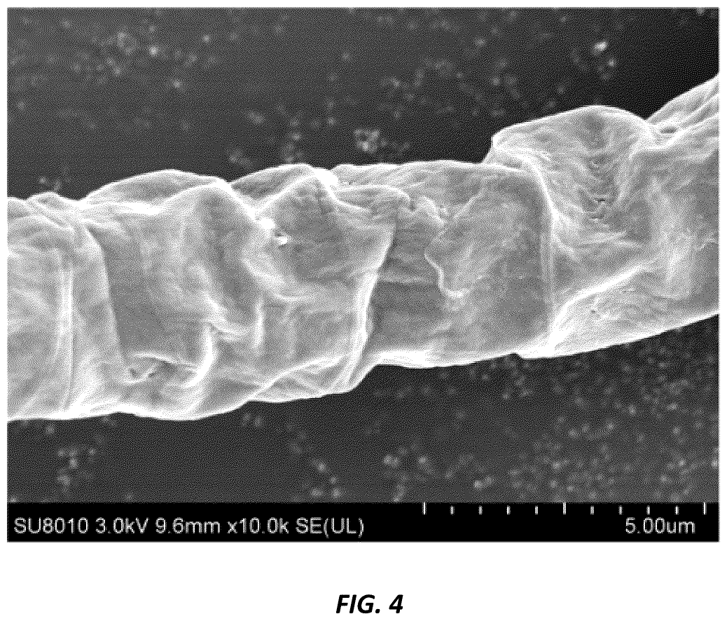

FIG. 4 shows an SEM image of the anti-radiation microalgae (Spirulina)-nanoparticle compound preparation in Example 2, in which a surface of the Spirulina is smooth and not covered by the nanoparticles. This indicates that it is difficult to combine nanoparticles having no modified surfaces with the surface of Spirulina, thereby impossible to effectively construct a microalgae-nanoparticle compound preparation.

### Example 3

A method for preparing an oral anti-radiation microalgae-nanoparticle compound preparation was performed according to the following procedures.
(1) Astaxanthin and PLGA were dissolved in dichloromethane (a mass ratio of the astaxanthin to the PLGA being 1:10). 2% polyvinyl alcohol aqueous solution was then added thereto, and a resulting mixture was stirred by ultrasonically oscillating with a probe at 4° C. for 5 min, forming an emulsion. The emulsion was stirred at room temperature for 12 h to volatilize the dichloromethane, initially forming astaxanthin-loaded nanoparticles.
(2) A dopamine solution was added to a solution of nanoparticles prepared in step (1), and a pH value of a resulting mixture was adjusted to 8.5. A resulting mixture was stirred at room temperature for 12 h. Excess unreacted materials were removed by washing, obtaining astaxanthin-loaded nanoparticles having modified surfaces.
(3) Spirulina was added into the aqueous solution containing the surface-modified drug-loaded nanoparticles obtained in step (2), wherein a mass ratio of the Spirulina to the drug-loaded nanoparticles being 10:1. A resulting mixture was stirred at room temperature for 12 h, and centrifugated. A resulting precipitate was collected, and excess unreacted materials were removed by washing, obtaining the oral anti-radiation microalgae-nanoparticle compound preparation, in which the microalgae and the drug-loaded nanoparticles were combined.

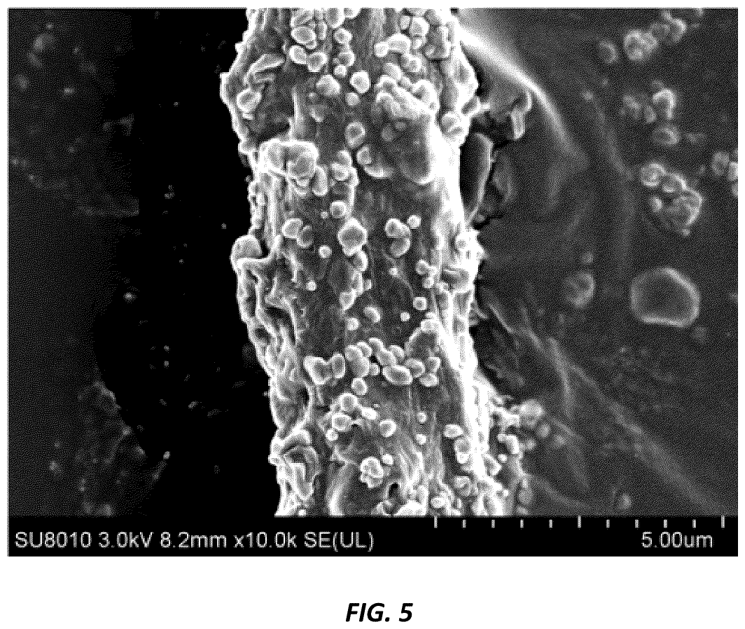

FIG. 5 shows an SEM image of the anti-radiation microalgae (Spirulina)-nanoparticle compound preparation in Example 3, in which a surface of the Spirulina is covered by a small number of the nanoparticles. This indicates that the nanoparticles having surfaces modified with dopamine are only partially combined with the surface of Spirulina, making it difficult to effectively construct a microalgae-nanoparticle compound preparation.

### Example 4

A method for preparing an oral anti-radiation microalgae-nanoparticle compound preparation was performed according to the following procedures.
(1) Astaxanthin was fully dissolved in dimethyl sulfoxide, and diluted with distilled water in a volume 10 times than that of dimethyl sulfoxide, obtaining an astaxanthin solution.
(2) Spirulina was added into the astaxanthin solution prepared in step (1), wherein a mass ratio of the Spirulina to the astaxanthin was 10:1. A resulting mixture was stirred at room temperature for 12 h, and centrifugated. A resulting supernatant was collected, and concentration changes of the astaxanthin were detected; a precipitate was collected, and excess unreacted materials were removed by washing, obtaining an astaxanthin-loaded microalgae.

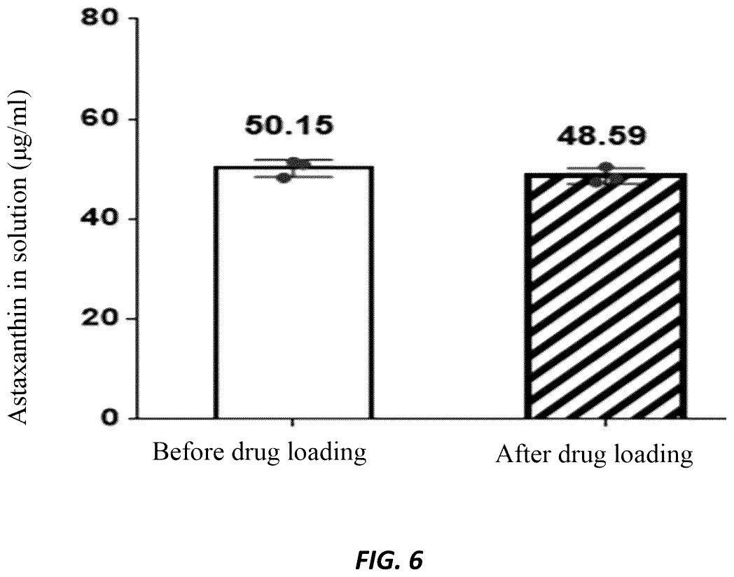

FIG. 6 shows concentrations of astaxanthin in a solution before and after drug loading in Example 4. As shown in FIG. 6, there is no statistical difference between the two concentrations (p value=0.3), indicating that astaxanthin in the solution is not loaded on the Spirulina.

The above embodiments are only some embodiments of the present disclosure. In the present disclosure, the anti-radiation drug is not limited to the astaxanthin, but can also be resveratrol, vitamin E, lycopene, zeaxanthin, curcumin, epigallocatechin gallate, and amifostine, etc. Any anti-radiation pharmaceutical component is acceptable as long as it can prevent and/or treat radiation-induced cell/tissue/organ damages. The method according to the present disclosure provides a feasible and effective loading method for poorly water-soluble or water-insoluble drugs such as astaxanthin, such that an oral anti-radiation compound preparation is formed from these drugs. The compound preparation makes it possible to improve water-solubility and oral absorbability of the anti-radiation drug, and realizes long-term retention in the intestinal tract, multi-stage slow release of the drug, and gradual degradation, which effectively enhances distribution and bioavailability of the drug in the intestinal tract, and has a beneficial regulatory effect on intestinal flora and metabolites thereof, thereby providing effective radiation protection for the intestinal tract and the whole body. Moreover, the anti-radiation compound preparation has a highly excellent therapeutic effect and can replace existing conventional drugs.

1. An oral anti-radiation microalgae-nanoparticle compound preparation, comprising a microalgae, an anti-radiation drug, and nanoparticles;
wherein the anti-radiation drug is loaded on the nanoparticles to form drug-loaded nanoparticles, and the drug-loaded nanoparticles are loaded on a surface of the microalgae through a surface modifier to form the oral anti-radiation microalgae-nanoparticle compound preparation for direct oral administration.

2. The oral anti-radiation microalgae-nanoparticle compound preparation as claimed in claim 1, wherein the anti-radiation drug comprises any ingredient capable of preventing and/or treating a damage to a cell, a tissue, or an organ caused by radiation.

3. The oral anti-radiation microalgae-nanoparticle compound preparation as claimed in claim 1, wherein the nanoparticles are at least one selected from the group consisting of poly(lactic-co-glycolic acid) nanoparticles, liposome nanoparticles, porous silica nanoparticles, porous carbon nanoparticles, dendrimer nanoparticles, and metal nanoparticles.

4. The oral anti-radiation microalgae-nanoparticle compound preparation as claimed in claim 1, wherein the surface modifier is one or more selected from the group consisting of chitosan, polyethyleneimine, guar gum, dopamine, sodium hyaluronate, polyvinyl alcohol, sodium alginate, calcium alginate, gelatin, and a cellulose derivative.

5. A method for preparing the oral anti-radiation microalgae-nanoparticle compound preparation as claimed in claim 1, comprising the steps of
mixing the anti-radiation drug with the nanoparticles in a solution, such that the nanoparticles encapsulate the anti-radiation drug after an organic agent is volatilized to obtain the drug-loaded nanoparticles;
adding the surface modifier into a first resulting solution containing the drug-loaded nanoparticles, mixing and performing reaction to obtain surface-modified drug-loaded nanoparticles;
adding the microalgae into a second resulting solution containing the surface-modified drug-loaded nanoparticles; and
mixing to obtain the oral anti-radiation microalgae-nanoparticle compound preparation.

6. The method as claimed in claim 5, wherein the solution is one or more selected from the group consisting of dichloromethane, acetone, ethanol, methanol, and a water-soluble solution, the water-soluble solution being one or more selected from the group consisting of water, a phosphate buffer, a citrate buffer, an acetate buffer, and a tris(hydroxymethyl) aminomethane hydrochloride buffer.

7. An oral anti-radiation medicine, comprising the oral anti-radiation microalgae-nanoparticle compound preparation as claimed in claim 1, wherein the anti-radiation refers to prevention, treatment, or alleviation of a damage or a disease in intestinal tract and other organs of a whole body caused by ionizing radiation or a radioactive substance.

8. The oral anti-radiation drug as claimed in claim 7, further comprising a pharmaceutically acceptable excipient.
